(19) United States
(12) Patent Application Publication
(10) Pub. No.: US 2018/0036411 A1
(43) Pub. Date: Feb. 8, 2018

(54) Stable Formulation of Insulin Glulisine

(71) Applicant: Sanofi, Paris (FR)

(72) Inventors: Petra Loos, Frankfurt am Main (DE); Thomas Gehrmann, Frankfurt am Main (DE); Harald Berchtold, Frankfurt am Main (DE); Ulrich Werner, Frankfurt am Main (DE); Matthias Ganz, Basel (CH)

(21) Appl. No.: 15/656,839

(22) Filed: Oct. 27, 2017

Related U.S. Application Data
(63) Continuation of application No. 14/523,842, filed on Oct. 24, 2014, now abandoned.

(30) Foreign Application Priority Data
Oct. 25, 2013 (EP) 13306475.8

(51) Int. Cl. A61K 47/20; A61K 9/00; A61K 38/28; A61K 47/18

(52) U.S. Cl. CPC A61K 47/20; A61K 47/18; A61K 38/28; A61K 9/0019

(57) ABSTRACT

An aqueous pharmaceutical formulation comprising 200-1000 U/mL of insulin glulisine.

The present invention relates to an aqueous pharmaceutical formulation comprising 200-1000 U/mL of insulin glulisine with improved stability, and its use in the treatment of type 1 diabetes mellitus or type 2 diabetes mellitus.

Worldwide, approximately 300 million people suffer from type 1 and type 2 diabetes mellitus. For type 1 diabetics the substitution of the lacking endocrine insulin secretion is the only currently possible therapy. The affected persons are dependent lifelong on insulin injections, as a rule a number of times daily. In contrast to type 1 diabetes, there is not basically a deficiency of insulin in type 2 diabetes, but in a large number of cases, especially in the advanced stage, treatment with insulin, optionally in combination with an oral antidiabetic, is regarded as the most favorable form of therapy.

In the healthy person, the release of insulin by the pancreas is strictly coupled to the concentration of the blood glucose. Elevated blood glucose levels, such as occur after meals, are rapidly compensated by a corresponding increase in insulin secretion. In the fasting state, the plasma insulin level falls to a basal value which is adequate to guarantee a continuous supply of insulin-sensitive organs and tissue with glucose and to keep hepatic glucose production low in the night. Often, the replacement of the endogenous insulin secretion by exogenous, mostly subcutaneous administration of insulin does not achieve the quality of the physiological regulation of the blood glucose described above. Deviations of the blood glucose upward or downward can occur, which in their severest forms can be life-threatening. In addition, blood glucose levels which are increased for years without initial symptoms are a considerable health risk. The large-scale DCCT study in the USA (The Diabetes Control and Complications Trial Research Group (1993) N. Engl. J. Med. 329, 977-986) demonstrated clearly that chronically elevated blood glucose levels are essentially responsible for the development of diabetic late complications, such as microvascular and macrovascular damage which is manifested, under certain circumstances, as retinopathy, nephropathy or neuropathy and leads to loss of sight, kidney failure and the loss of extremities. Moreover diabetes is accompanied by an increased risk of cardiovascular diseases. It is to be derived from this that an improved therapy of diabetes is primarily to be aimed at keeping the blood glucose as closely as possible in the physiological range. According to the concept of intensified insulin therapy, this should be achieved by repeated daily injections of rapid- and slow-acting insulin preparations. Rapid-acting formulations are given at meals in order to level out the postprandial increase in the blood glucose. Slow-acting basal insulins should ensure the basic supply with insulin, in particular during the night, without leading to hypoglycemia.

Insulin is a polypeptide of 51 amino acids, which are divided into 2 amino acid chains: the A chain having 21 amino acids and the B chain having 30 amino acids. The chains are connected to one another by means of 2 disulfide bridges. Insulin preparations have been employed for diabetes therapy for many years. Not only naturally occurring insulins are used here, but recently also insulin derivatives and analogs.

The insulin preparations of naturally occurring insulins on the market for insulin substitution differ in the origin of the insulin (e.g. bovine, porcine, human insulin, or another mammalian or animal insulin), and also the composition, whereby the profile of action (onset of action and duration of action) can be influenced. By combination of various insulin preparations, very different profiles of action can be obtained and blood sugar values which are as physiological as possible can be established. Preparations of naturally occurring insulins, as well as preparations of insulin derivatives or insulin analogs which show modified kinetics, have been on the market for some time. Recombinant DNA technology today makes possible the preparation of such modified insulins. These include “monomeric insulin analogs” such as insulin lispro, insulin aspart, and HMR 1964 (Lys(B3), Glu(B29) human insulin, insulin glulisine), all of which have a rapid onset of action, as well as insulin glargin, which has a prolonged duration of action.

In addition to the duration of action, the stability of the preparation is very important for patients. Stabilized insulin formulations having increased physical long-term stability are needed in particular for preparations which are exposed to particular mechanical stresses or relatively high temperatures. These include, for example, insulins in administration systems such as pens, inhalation systems, needleless injection systems or insulin pumps. Insulin pumps are either worn on or implanted in the body of the patient. In both cases, the preparation is exposed to the heat of the body and movement and to the delivery motion of the pump and thus to a very high thermomechanical stress. Since insulin pens too (disposable and reutilizable pens) are usually worn on the body, the same applies here. Previous preparations have only a limited stability under these conditions.

Insulin is generally present in neutral solution in pharmaceutical concentration in the form of stabilized zinc-containing hexamers, which are composed of 3 identical dimer units (Brange et al., Diabetes Care 13:923-954 (1990)). However, the profile of action of an insulin preparation may be improved by reducing the oligomeric state of the insulin it contains. By modification of the amino acid sequence, the self-association of insulin can be decreased. Thus, the insulin analog lispro, for example, mainly exists as a monomer and is thereby absorbed more rapidly and shows a shorter duration of action (HPT Ammon and C. Werning; Antidiabetika [Antidiabetics]; 2. Ed.; Wiss. Verl.-Ges. Stuttgart; 2000; p. 94.f). However, the rapid-acting insulin analogs which often exist in the monomeric or dimeric form are less stable and more prone to aggregate under thermal and mechanical stress than hexameric insulin. This makes itself noticeable in cloudiness and precipitates of insoluble aggregates. (Bakaysa et al, U.S. Pat. No. 5,474,978). These higher molecular weight transformation products (dimers, trimers, polymers) and aggregates decrease not only the dose of insulin administered but can also induce irritation or immune reactions in patients. Moreover, such insoluble aggregates can affect and block the cannulas and tubing of the pumps or needles of pens. Since zinc leads to an additional stabilization of insulin through the formation of zinc-containing hexamers, zinc-free or low-zinc preparations of insulin and insulin analogs are particularly susceptible to instability. In particular, monomeric insulin analogs having a rapid onset of action are prone to aggregate and become physically unstable very rapidly, because the formation of insoluble aggregates proceeds via monomers of insulin.

In order to guarantee the quality of an insulin preparation, it is necessary to avoid the formation of aggregates. There are various approaches for stabilizing insulin formulations. Thus, in international patent application WO98/56406, formulations stabilized by Tris or arginine buffer have been described. U.S. Pat. No. 5,866,538 describes an insulin preparation which contains glycerol and sodium chloride in concentrations of 5-100 mM and should have an increased stability. U.S. Pat. No. 5,948,751 describes insulin preparations having increased physical stability, which is achieved by addition of mannitol or similar sugars. The addition of excess zinc to a zinc-containing insulin solution can likewise increase the stability (J. Brange et al., Diabetic Medicine, 3: 532-536, 1986). The influence of the pH and various excipients on the stability of insulin preparations has also been described in detail (J. Brange & L. Langkjaer, Acta Pharm. Nordica 4: 149-158).

Often, these stabilization methods are not adequate for increased demands (improvement in ability to be kept at room or body temperature and under mechanical stress) or for “monomeric” insulin analogs or rapid-acting insulins, which are particularly susceptible to physical stress. Moreover, all commercial insulin preparations contain zinc, which is added to stabilize the preparation. Thus, Bakaysa et al. in U.S. Pat. No. 5,474,978 describe stabilized formulations of insulin complexes which consist of 6 insulin analog monomers, 2 zinc atoms and at least 3 molecules of a phenolic preservative. These formulations can additionally contain a physiologically acceptable buffer and a preservative. If it is wished, however, to prepare zinc-free or low-zinc insulin preparations, the stabilization methods mentioned are not adequate for a marketable preparation. For example, it was not possible to develop a zinc-free preparation of insulin lispro on account of inadequate physical stability (Bakaysa et al., Protein Science (1996), 5:2521-2531). Low-zinc or zinc-free insulin formulations having adequate stability, in particular physical stability, are not described in the prior art.

Zinc-free formulations of insulin glulisine can be stabilized by surfactants. WO 02/076495 discloses an U100 insulin glulisine (100 IU/ml) formulation containing polysorbate 20, polysorbate 80 or poloxamer 171.

The problem of the present invention can be seen in the provision of a pharmaceutical formulation of insulin glulisine overcoming at least partially the above-described stability issues, wherein potentially disadvantageous components should be avoided. In particular, the problem of the present invention can be seen in the provision of a pharmaceutical formulation of insulin glulisine having an improved stability at elevated temperature (such as the body temperature).

In the present invention, it was surprisingly found that the physical long-term stability of a formulation containing 200-1000 U/mL insulin glulisine is increased, in particular at elevated temperatures.

By the improved physical stability at elevated temperatures, the formulations as described herein are suitable for administration by devices implanted into the patient or otherwise exposed to the body temperature. For example, the formulation of the present invention is suitable for use in insulin pumps implanted in the patient's body or in patch pumps worn close to the body. Furthermore, the formulation is suitable for use in injection devices, such as pens, syringes, injectors, or for any use in which increased physical stability at elevated temperature is necessary, for example if these devices are worn close to the body.

If, for example, an U300 formulation of insulin glulisine comprising trometamol, glycerol and phenol is administered instead of an U100 formulation, the volume to be administered can be reduced. The reduced volume, together with the improved stability, improves administration by an insulin pump or a patch pump, as the pump can be used for a longer time without replacement of the reservoir, or/and the size of the pump can be reduced.

In an animal model, surprisingly no difference in pharmacokinetics and pharmacodynamics was detected in U100 and U300 formulations of insulin glulisine.

In the present invention, insulin glulisine is Lys(B3), Glu(B29) human insulin. Insulin glulisine has a molecular weight of 5823 Dalton. A 0.6 mM solution of insulin glulisine contains 3,4938 mg/mL insulin glulisine (100 units/mL, U100). An U300 insulin glulisine formulations contains 300 U/mL insulin glulisine (10.4814 mg/mL or 10.48 mg/mL).

As used herein, the term “stability” refers to the chemical and/or physical stability of active pharmaceutical ingredients, in particular of insulin analogues and/or derivatives. The purpose of stability testing is to provide evidence on how the quality of an active pharmaceutical ingredient or dosage form varies with time under the influence of a variety of environmental factors such as temperature, humidity, and light, and to establish a shelf life for the active pharmaceutical ingredient or dosage form and recommended storage conditions. Stability studies should include testing of those attributes of the active pharmaceutical ingredient that are susceptible to change during storage and are likely to influence quality, safety, and/or efficacy. The testing should cover, as appropriate, the physical, chemical, biological, and microbiological attributes, preservative content (e.g., antioxidant, antimicrobial preservative), and functionality tests (e.g. for a dose delivery system). Analytical procedures should be fully validated and stability indicating. In general, significant changes for an active pharmaceutical ingredient and/or dosage form with regard to stability are defined as:
a 5% change in assay from its initial value; or failure to meet the acceptance criteria for potency when using biological or immunological procedures;
any degradation products exceeding its acceptance criterion;
failure to meet the acceptance criteria for appearance, physical attributes, and functionality test (e.g., color, phase, separation, resuspendibility, caking, hardness, dose delivery per actuation); however, some changes in physical attributes (e.g. softening of suppositories, melting of creams) may be expected under accelerated conditions;
and, as appropriate for the dosage form:
failure to meet the acceptance criterion for pH; or
failure to meet the acceptance criteria for dissolution for 12 dosage units.

The significant changes may also be evaluated against established acceptance criteria prior to starting the evaluation of the stability.

Acceptance criteria should be derived from the monographs (e.g. monographs for the European Pharmacopeia, of the United States Pharmacopeia, of the British Pharmacopeia, or others), and from the analytical batches of the active pharmaceutical ingredient and medicinal product used in the preclinical and clinical studies. Acceptable limits should be proposed and justified, taking into account the levels observed in material used in preclinical and clinical studies. Product characteristics may be visual appearance, purity, color and clarity for solutions/suspensions, visible particulates in solutions, and pH. For example, suitable acceptance criteria for insulin glulisine formulations during shelf life are linked with the test items: Appearance of solution (visual), assay insulin glulisine (HPLC), related impurities (HPLC), high molecular weight proteins (HPSEC), particulate matter (visible particles), particulate matter (subvisible particles), assay m-cresol and phenol, zinc (AAS).

The acceptance criteria and/or test items shown above are based on monographed acceptance limits and/or are derived from extensive experience in the development of insulin formulations.

As used herein, the term “treatment” refers to any treatment of a mammalian, for example human condition or disease, and includes: (1) inhibiting the disease or condition, i.e., arresting its development, (2) relieving the disease or condition, i.e., causing the condition to regress, or (3) stopping the symptoms of the disease.

As used herein, the unit of measurement “U” and/or “international units” refers to the blood glucose lowering activity of insulin and is defined (according to the World Health Organization, WHO) as follows: 1 U corresponds to the amount of highly purified insulin (as defined by the WHO) which is sufficient to lower the blood glucose level of a rabbit (having a body weight of 2-2.5 kg) to 50 mg/100 mL within 1 hour and to 40 mg/100 mL within 2 hours. For human insulin, 1 U corresponds to approximately 35 μg (Lill, Pharmazie in unserer Zeit, No. 1, pp. 56-61, 2001). For insulin glulisine, 100 U correspond to 3.49 mg (product information Apidra® cartridges).

An embodiment of the invention is an aqueous pharmaceutical formulation comprising 200-1000 U/mL of insulin glulisine, more specifically such formulations comprising insulin glulisine in a concentration of 200, 250, 300, 350, 400, 450, 500, 550, 600, 650, 700, 750, 800, 850, 900, 950 or 1000 U/ml. A further embodiment of the invention is an aqueous pharmaceutical formulation comprising 200-500 U/mL of insulin glulisine, more specifically 270-330 U/mL of insulin glulisine, further preferred 300 U/mL of insulin glulisine.

An embodiment of the invention is an aqueous pharmaceutical formulation as described above which is essentially free of zinc or contains 20 μg/mL of zinc or less.

An embodiment of the invention is an aqueous pharmaceutical formulation as described above comprising at least one substance selected from buffer substances, preservatives, and tonicity agents, preferably wherein the buffer substance is trometamol.

An embodiment of the invention is an aqueous pharmaceutical formulation as described above comprising 3 to 10 mg/mL of trometamol.

An embodiment of the invention is an aqueous pharmaceutical formulation as described above, wherein the preservative is phenol and/or m-cresol.

An embodiment of the invention is an aqueous pharmaceutical formulation as described above comprising 1.5 to 3.5 mg/mL of m-cresol and/or 0.5 to 3.0 mg/ml of phenol.

An embodiment of the invention is an aqueous pharmaceutical formulation as described above, wherein the tonicity agent is glycerol.

An embodiment of the invention is an aqueous pharmaceutical formulation as described above, comprising 5 to 26 mg/mL of glycerol.

An embodiment of the invention is an aqueous pharmaceutical formulation as described above claims which is essentially free of phosphate.

An embodiment of the invention is an aqueous pharmaceutical formulation as described above comprising an amino acid selected from a group comprising arginine and methionine, in particular in a concentration from 1 to 30 mg/ml.

An embodiment of the invention is an aqueous pharmaceutical formulation as described above comprising a non-ionic surfactant, wherein the non-ionic surfactant is preferably selected from a group comprising polysorbate 20, polysorbate 80 and poloxamer 171.

An embodiment of the invention is an aqueous pharmaceutical formulation as described above, wherein the non-ionic surfactant is present in a concentration of 1 to 200 μg/ml, preferably 10 to 20 μg/ml, and more preferred 10 μg/ml.

An embodiment of the invention is an aqueous pharmaceutical formulation as described above, wherein the pH is between 3.5 and 9.5, preferably between 6 and 8.5 and more preferred between 7 and 7.8.

A further embodiment of the invention is a medical device comprising the formulation as described above. Such medical device can be an insulin pump or a pen for injection.

The aqueous pharmaceutical formulation of any of the foregoing claims which is essentially free of chloride. An essentially free of chloride formulation of the invention can, however, a low amount from chloride that is added to the formulation solely for the purpose of pH adjustment.

An embodiment of the invention is an aqueous pharmaceutical formulation as described above, for injection.

An embodiment of the invention is an aqueous pharmaceutical formulation as described above, for administration by an insulin pump.

An embodiment of the invention is an aqueous pharmaceutical formulation as described above, for use in the treatment of type 1 diabetes mellitus or type 2 diabetes mellitus.

An embodiment of the invention is a method of treatment of type 1 diabetes mellitus or type 2 diabetes mellitus, comprising administration of the formulation as described above to a patient suffering from type 1 diabetes mellitus or type 2 diabetes mellitus, preferably wherein the formulation is administered by injection or by an insulin pump.

An embodiment of the invention is the use of a formulation as described above for the manufacture of a medicament for the treatment of type 1 diabetes mellitus or 2 diabetes mellitus.

As mentioned above, the aqueous pharmaceutical formulation of the present invention can contain a surfactant. Suitable pharmaceutically acceptable surfactants are disclosed in WO 02/076495, the disclosure of which is included herein by reference. In particular, the surfactant is selected from polysorbate 20, polysorbate 80 and poloxamer 171. The surfactant, in particular polysorbate 20, can be present in an amount of 1 to 200 μg/mL, preferably 10 to 20 μg/mL, and more preferred 10 μg/mL.

The buffer substance can be selected from pharmaceutically acceptable buffer substances, such as phosphate or trometamol. Phosphate dihydrate can be present in an amount of 1 to 5 mg/mL. A preferred buffer substance is trometamol (Tris, tris(hydroxymethyl)-aminomethan), which can be present in the formulation in a concentration of 3 to 10 mg/mL, preferably 6 mg/mL.

It is preferred that the formulation of the present invention is suitable for parenteral administration. The formulation can be injected or administered by an insulin pump or a pen. The insulin pump can be a patch pump. The skilled person knows suitable devices.

The aqueous pharmaceutical formulation of the present invention is for use in the treatment of a patient suffering from type 1 diabetes mellitus or type 2 diabetes mellitus. The patient is in particular a human.

Another aspect of the present invention is a method of treatment of type 1 diabetes mellitus or type 2 diabetes mellitus, comprising administration of an aqueous pharmaceutical formulation of the present invention to a patient suffering from type 1 diabetes mellitus or type 2 diabetes mellitus. The formulation is preferably administered by injection, an insulin pump or a pen. The patient is in particular a human.

Yet another aspect of the present invention is the use of an aqueous pharmaceutical formulation of the present invention for the manufacture of a medicament for the treatment of type 1 diabetes mellitus or 2 diabetes mellitus.

The invention is further described by the following figures and examples.

### LEGENDS

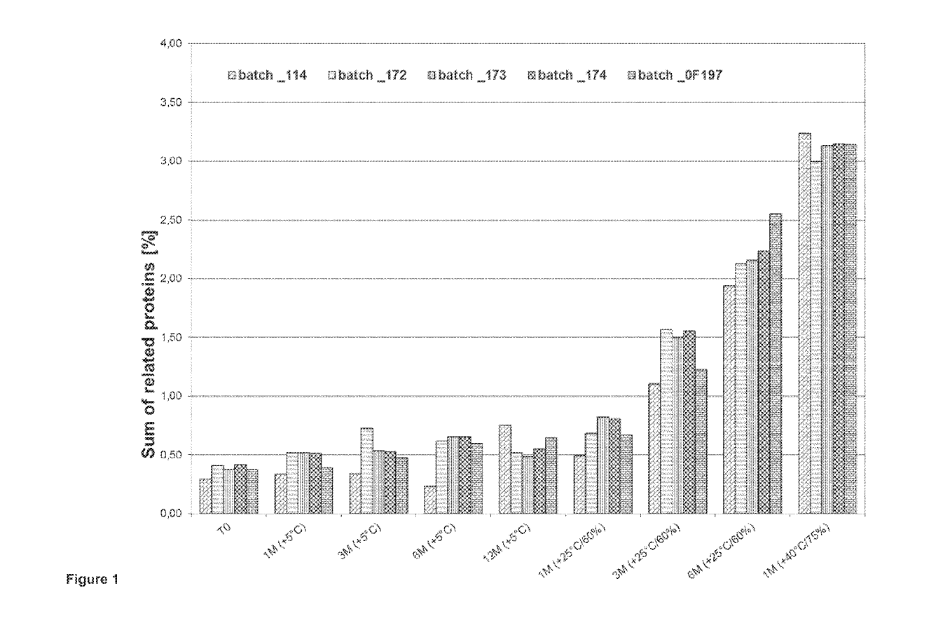

FIG. 1: Sum of related proteins for U300 formulations according to the invention (_114, _172, _173, _174)

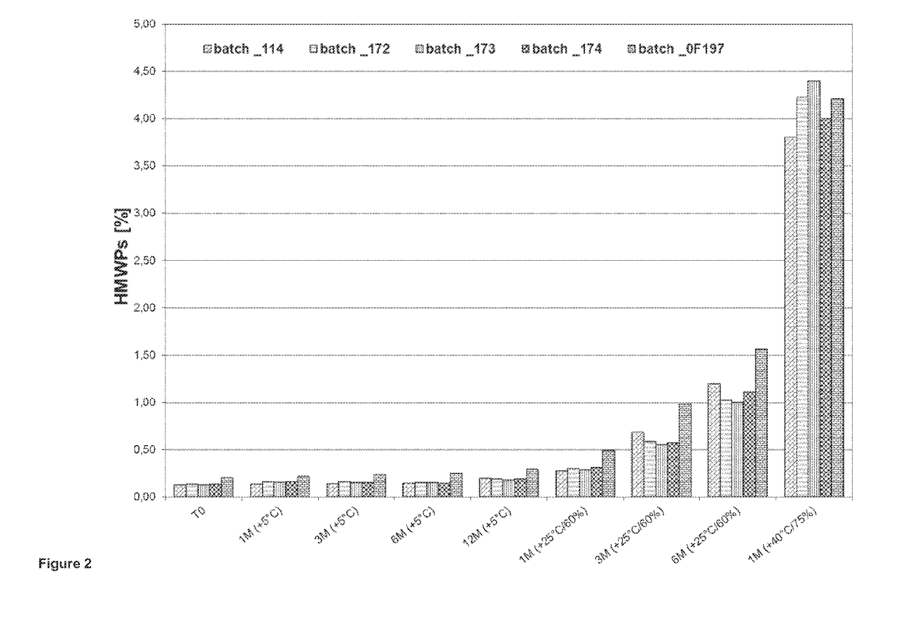

FIG. 2: High molecular weight proteins for U300 formulations according to the invention (_114, _172, _173, _174).

### EXAMPLE 1

U300 Insulin glulisine formulations stored at different conditions were compared in terms of high molecular weight proteins (HMWPs), the content of insulin glulisine, the increase of related proteins and visual clarity.

An U300 insulin glulisine formulation contains 300 U/mL insulin glulisine (10.48 mg/mL).

The content of HMPWs describes the degree of aggregation of insulin molecules. Dimers, trimers and polymers of insulin can be observed. An increase of HMPWs indicates a larger proportion of insulin molecules being aggregated.

The results indicate that the excipients have an impact upon the stability of an U300 formulation of insulin glulisine at elevated temperature and under the influence of light. In particular, the presence of methionine or a small amount of zinc can stabilize the U300 insulin glulisine formulation at elevated temperature and under the influence of light.

A systematic comparison of the influence of excipients on stability of U100 and U300 formulations of insulin glulisine formulations was performed. Sixteen U300 and U100 formulations were prepared, representing all permutations of
tonicity agent glycerol or NaCl
preservative m-cresol or phenol
buffer substance trometamol or phosphase dihydrate
surfactant polysorbate 20 or no surfactant

By this approach, the stability of formulations being different in only one of these four components can be compared.

Additional formulations were prepared to compare the effect of the presence of zinc upon the stability.

The formulations were stored at 37° C. for 30 days and physical and chemical stability was assessed.

### EXAMPLE 2

### Control of the Formulation

### (a) Analytical Procedures

Tests are carried out using compendial analytical test methods, where applicable. The quality control concept has been established taking into account the cGMP requirements as well as the current status of the ICH process.

The non-compendial and chromatographic analytical procedures used to control the formulation are summarized in the following:

### Description

Visually examine a number of containers for conformance to the acceptance criteria.

### Identification (HPLC)

The identity of the active ingredient is ensured by comparing the retention time of the drug formulation sample with the retention time of the reference standard using a reversed phase HPLC method. The method is also used for the determination of assay of the active ingredient, for the determination of the related compounds and impurities, and for quantifying the preservatives m-cresol and phenol.

### Assay (HPLC)

The test is carried out by reverse phase liquid chromatography (HPLC). The method is also used for the identification, the determination of assay of the active ingredient, for the determination of the related compounds and impurities, and for quantifying the preservatives m-cresol and phenol. Column: Octadecylsilylated silica gel (C18), particle size 3 μm, pore size 200 Å (250 mm×4.0 mm), thermostated at +41° C. Autosampler: Thermostated at ≦+10° C. Mobile phase A: Buffer solution pH 2.2/acetonitrile/water (55:20:25 v/v). Mobile phase B: Buffer solution pH 2.2/acetonitrile (55:45 v/v). Gradient is shown in Table 1.

TABLE 1
HPLC gradient

| Time [min] | Mobile phase A [%] | Mobile phase B [%] |
|---|---|---|
| 0 | 67.5 | 32.5 |
| 0 to 42 | 67.5 | 32.5 |
| 42 to 70 | 40 | 60 |
| 70 to 75 | 40 | 60 |
| 76 to 76 | 67.5 | 32.5 |
| 76 to 90 | 67.5 | 32.5 |

Flow rate: 0.6 mL/min. Injection volume: 12 μL. Detection: 205 nm (for the active ingredient) and 252 nm (for m-cresol and phenol). Typical run time: 90 min. Assay of the active ingredient, m-cresol and phenol are calculated by external standardization. Impurities are calculated using the peak area percent method.

### Related Compounds and Impurities (HPLC)

The same chromatographic conditions as for “Assay (HPLC)” are used for the determination of related compounds and impurities. Related compounds and Impurities are calculated using the peak area percent method.

### High Molecular Weight Proteins (HMWPs)

The high molecular weight proteins are determined using high pressure size exclusion chromatography (HPSEC). Column: Shodex Protein KW 802.5 (silica gel, diol) 120-7-diol, separating range 2000 to 80000 Daltons (300 mm×8 mm), thermostated at room temperature. Autosampler: Thermostated at ≦+10° C. Mobile phase: Acetic acid/acetonitrile/water (200:300:400 v/v), adjusted to pH 3.0 with ammonia solution 25% (v/v). Isocratic elution. Flow rate: 0.5 mL/min. Injection volume: 100 μL. Detection: 276 nm. Typical run time: 65 min.

HMWPs are calculated using the peak area percent method.

### Antimicrobial Preservative Assay

The same chromatographic conditions as for “Assay (HPLC)” are used for the determination of assay of m-Cresol and of phenol. m-cresol and phenol are calculated by external standardization.

### (c) Justification of the Acceptance Criteria

Tests and acceptance criteria, as previously presented, were selected based on ICH Q6B and on published monographs, analytical results obtained, precision of procedures used, Pharmacopoeial and/or regulatory guidelines, and are in agreement with the standard limits at this stage of development.

### Feasibility of the Formulation to Form Concentrated Solutions

Formulations from 100 to 900 Units/mL were included to investigate the feasibility of insulin glulisine concentrated solutions. The maximum solubility in water at the intended pH of 7.3 was determined to be around 1100 Units/mL. The chemical and physical stability of formulations from 100 to 900 Units/mL can be confirmed.

### Stability of the Formulation

### (a) Stability of the Formulation

Stability studies for the formulation were initiated according to the stability protocol summary described in the following table. The composition and manufacturing method of the stability batches is representative of the material. The stability profile was assessed for storage under long term, accelerated, and stress testing conditions according to ICH guidelines. Samples were packed and stored in 3 mL cartridges with flanged aluminum cap and inserted laminated sealing disc. Up to now, 12 months stability data are available from ongoing stability studies of the formulation.

TABLE 2
Storage Conditions

| Storage Condition | Sampling Intervals | | Container |
|---|---|---|---|
| Long Term | | | |
| +5° C. ± 3° C. | 1, 3, 6, and 12 | months | 3 mL cartridges |
| Accelerated | | | |
| +25° C. ± 2° C./60% ± 5% | 1, 3, and 6 | months | 3 mL cartridges |
| RH | | | |
| Stress | | | |
| +40° C. ± 5° C./75% ± 5% | 1 | month | 3 mL cartridges |
| RH | | | |
| Photostability | | | |
| Sun test according to ICH | 1 | day | 3 mL cartridges |
| guidelines* | | | |
| Indoor light** | 14 | days | 3 mL cartridges |

*Overall illumination of not less than 1.2 million lux hours and an integrated near ultraviolet energy of not less than 200 watt hours/m2. A dark control sample is stored under the same conditions to eliminate any effects due to local temperature changes
**Variolux, Heraeus, standard fluorescent tubes, GE-Lightening, Type F40/33, irradiance approximately 8 W/m2, 2000 Lux. A dark control sample is stored under the same conditions to evaluate any effects due to local temperature changes

The following tests were performed during stability testing: appearance, assay, related proteins, high molecular weight proteins, pH, particulate matter (visible and subvisible particles), assay of antimicrobial preservatives (m-cresol and phenol), content of zinc. The investigations on physical properties after 6 months of storage at the long term storage condition of +5° C. and chemical properties after 12 months of storage at the long term storage condition of +5° C. confirm the stability of the formulation when stored at the recommended storage condition. Only very slight changes of the related impurities could be observed.

When stored at accelerated conditions (6 months at +25° C./60% RH) the related proteins and high molecular weight proteins increased slightly. When stored at accelerated conditions (1 month at +40° C./75% RH) the related proteins and high molecular weight proteins increased. The content of the active ingredient, m-cresol and phenol remained basically unchanged under accelerated conditions.

Due to the present results of the stability studies of the formulation, the chemical and physical stability of the formulation can be confirmed.

Tables 3 and 4 show the long term, accelerated and stress stability results, wherein batch nos. “_114”, “_172”, “_173” and “_174” are referring to a formulation according to the present invention.

TABLE 3
Comparison of chemical stability of the U300 formulations according to the invention (_114, _172, _173, _174)
against other U300 formulations

| | | | Composition | | | | | | | |
|---|---|---|---|---|---|---|---|---|---|---|
| | | | Insulin | Sodium | | | | | Phosphate | |
| | Storage | Storage | glulisine | chloride | Glycerol | m-Cresol | Phenol | Trometamol | dihydrate | Polysorbate 20 |
| Formulation | condition | duration | [mg/mL] | [mg/mL] | [mg/mL] | [mg/mL] | [mg/mL] | [mg/mL] | [mg/mL] | [mg/mL] |
| 0F197 | +5° C. | 12 months | 3.49 | 5 | — | 3.15 | — | 6 | — | 0.01 |
| | +25° C./60% RH | 6 months | 3.49 | 5 | — | 3.15 | — | 6 | — | 0.01 |
| | +40° C./75% RH | 1 month | 3.49 | 5 | — | 3.15 | — | 6 | — | 0.01 |
| _114 | +5° C. | 12 months | 10.48 | — | 9.5 | 2.1 | 1.5 | 6 | — | 0.01 |
| | +25° C./60% RH | 6 months | 10.48 | — | 9.5 | 2.1 | 1.5 | 6 | — | 0.01 |
| | +40° C./75% RH | 1 month | 10.48 | — | 9.5 | 2.1 | 1.5 | 6 | — | 0.01 |
| _172 | +5° C. | 12 months | 10.48 | — | 13.2 | 2.1 | 1.5 | 6 | — | 0.01 |
| | +25° C./60% RH | 6 months | 10.48 | — | 13.2 | 2.1 | 1.5 | 6 | — | 0.01 |
| | +40° C./75% RH | 1 month | 10.48 | — | 13.2 | 2.1 | 1.5 | 6 | — | 0.01 |
| _173 | +5° C. | 12 months | 10.48 | — | 11.3 | 2.1 | 1.5 | 6 | — | 0.01 |
| | +25° C./60% RH | 6 months | 10.48 | — | 11.3 | 2.1 | 1.5 | 6 | — | 0.01 |
| | +40° C./75% RH | 1 month | 10.48 | — | 11.3 | 2.1 | 1.5 | 6 | — | 0.01 |
| _174 | +5° C. | 12 months | 10.48 | — | 10.1 | 3.15 | — | 6 | — | 0.01 |
| | +25° C./60% RH | 6 months | 10.48 | — | 10.1 | 3.15 | — | 6 | — | 0.01 |
| | +40° C./75% RH | 1 month | 10.48 | — | 10.1 | 3.15 | — | 6 | — | 0.01 |
| _105 | +5° C. | 12 months | 10.48 | 5 | — | 3.15 | — | 6 | — | 0.01 |
| | +25° C./60% RH | 6 months | 10.48 | 5 | — | 3.15 | — | 6 | — | 0.01 |
| | +40° C./75% RH | 1 month | 10.48 | 5 | — | 3.15 | — | 6 | — | 0.01 |
| _106 | +5° C. | 12 months | 10.48 | 0.58 | 16 | 1.72 | 1.5 | — | 1.25 | — |
| | +25° C./60% RH | 6 months | 10.48 | 0.58 | 16 | 1.72 | 1.5 | — | 1.25 | — |
| | +40° C./75% RH | 1 month | 10.48 | 0.58 | 16 | 1.72 | 1.5 | — | 1.25 | — |
| _107.1 | +5° C. | 12 months | 10.48 | 0.58 | 16 | 1.72 | 1.5 | — | 1.25 | — |
| | +25° C./60% RH | 6 months | 10.48 | 0.58 | 16 | 1.72 | 1.5 | — | 1.25 | — |
| | +40° C./75% RH | 1 month | 10.48 | 0.58 | 16 | 1.72 | 1.5 | — | 1.25 | — |
| _109 | +5° C. | 12 months | 10.48 | — | 10.2 | 2.1 | 1.5 | 6 | — | 0.01 |
| | +25° C./60% RH | 6 months | 10.48 | — | 10.2 | 2.1 | 1.5 | 6 | — | 0.01 |
| | +40° C./75% RH | 1 month | 10.48 | — | 10.2 | 2.1 | 1.5 | 6 | — | 0.01 |
| _111 | +5° C. | 12 months | 10.48 | — | 10.0 | 2.4 | 1.5 | 6 | — | 0.01 |
| | +25° C./60% RH | 6 months | 10.48 | — | 10.0 | 2.4 | 1.5 | 6 | — | 0.01 |
| | +40° C./75% RH | 1 month | 10.48 | — | 10.0 | 2.4 | 1.5 | 6 | — | 0.01 |
| _112 | +5° C. | 12 months | 10.48 | — | 10.6 | 2.4 | 0.9 | 6 | — | 0.01 |
| | +25° C./60% RH | 6 months | 10.48 | — | 10.6 | 2.4 | 0.9 | 6 | — | 0.01 |
| | +40° C./75% RH | 1 month | 10.48 | — | 10.6 | 2.4 | 0.9 | 6 | — | 0.01 |
| _113 | +5° C. | 12 months | 10.48 | — | 10.3 | 2.1 | 1.5 | 6 | — | 0.01 |
| | +25° C./60% RH | 6 months | 10.48 | — | 10.3 | 2.1 | 1.5 | 6 | — | 0.01 |
| | +40° C./75% RH | 1 month | 10.48 | — | 10.3 | 2.1 | 1.5 | 6 | — | 0.01 |

| | | | | | | Content | | | Sum of | |
|---|---|---|---|---|---|---|---|---|---|---|
| | | | Composition | | | Insulin | Content | Content | related | |
| | Storage | Storage | Zinc | Arginine | Methionine | glulisine | m-Cresol | Phenol | proteins | HMWPs |
| Formulation | condition | duration | [mg/mL] | [mg/mL] | [mg/mL] | [mg/mL] | [mg/mL] | [mg/mL] | [%] | [%] |
| 0F197 | +5° C. | 12 months | — | — | — | 3.52 | 3.14 | — | 0.65 | 0.29 |
| | +25° C./60% RH | 6 months | — | — | — | 3.67 | 3.07 | — | 2.55 | 1.57 |
| | +40° C./75% RH | 1 month | — | — | — | 3.41 | 3.06 | — | 3.15 | 4.21 |
| _114 | +5° C. | 12 months | — | — | 9 | 10.39 | 2.09 | 1.48 | 0.76 | 0.20 |
| | +25° C./60% RH | 6 months | — | — | 9 | 10.47 | 2.08 | 1.47 | 1.94 | 1.20 |
| | +40° C./75% RH | 1 month | — | — | 9 | 10.12 | 2.03 | 1.48 | 3.24 | 3.81 |
| _172 | +5° C. | 12 months | — | — | 3 | 10.64 | 2.03 | 1.46 | 0.52 | 0.19 |
| | +25° C./60% RH | 6 months | — | — | 3 | 10.20 | 1.99 | 1.44 | 2.13 | 1.02 |
| | +40° C./75% RH | 1 month | — | — | 3 | 9.95 | 2.13 | 1.53 | 3.00 | 4.23 |
| _173 | +5° C. | 12 months | — | — | 6 | 10.56 | 2.03 | 1.46 | 0.49 | 0.18 |
| | +25° C./60% RH | 6 months | — | — | 6 | 10.18 | 2.04 | 1.46 | 2.16 | 1.00 |
| | +40° C./75% RH | 1 month | — | — | 6 | 9.75 | 2.00 | 1.43 | 3.14 | 4.40 |
| _174 | +5° C. | 12 months | — | — | 9 | 10.74 | 3.01 | — | 0.56 | 0.19 |
| | +25° C./60% RH | 6 months | — | — | 9 | 10.06 | 3.04 | — | 2.24 | 1.11 |
| | +40° C./75% RH | 1 month | — | — | 9 | 9.59 | 3.00 | — | 3.15 | 4.00 |
| _105 | +5° C. | 12 months | — | — | — | 15.58 | 3.12 | — | 0.50 | 0.30 |
| | +25° C./60% RH | 6 months | — | — | — | 9.88 | 3.05 | — | 2.28 | 2.12 |
| | +40° C./75% RH | 1 month | — | — | — | 10.12 | 3.02 | — | 3.28 | 5.61 |
| _106 | +5° C. | 12 months | 0.0196 | — | — | 15.54 | 1.70 | 1.47 | 0.61 | 0.22 |
| | +25° C./60% RH | 6 months | 0.0196 | — | — | 9.64 | 1.63 | 1.42 | 1.83 | 1.60 |
| | +40° C./75% RH | 1 month | 0.0196 | — | — | 9.65 | 1.64 | 1.45 | 3.85 | 5.08 |
| _107.1 | +5° C. | 12 months | — | — | — | 10.47 | 1.70 | 1.47 | 0.83 | 0.43 |
| | +25° C./60% RH | 6 months | — | — | — | 9.78 | 1.68 | 1.46 | 1.98 | 2.26 |
| | +40° C./75% RH | 1 month | — | — | — | 9.80 | 1.66 | 1.48 | 3.98 | 5.59 |
| _109 | +5° C. | 12 months | 0.0196 | 9 | — | 10.56 | 2.09 | 1.48 | 0.78 | 0.25 |
| | +25° C./60% RH | 6 months | 0.0196 | 9 | — | 9.67 | 2.04 | 1.45 | 2.04 | 1.49 |
| | +40° C./75% RH | 1 month | 0.0196 | 9 | — | 9.92 | 2.05 | 1.49 | 3.41 | 5.93 |
| _111 | +5° C. | 12 months | — | 9 | — | 10.49 | 2.40 | 1.48 | 0.93 | 0.30 |
| | +25° C./60% RH | 6 months | — | 9 | — | 9.90 | 2.36 | 1.46 | 2.28 | 2.16 |
| | +40° C./75% RH | 1 month | — | 9 | — | 9.82 | 2.37 | 1.50 | 3.13 | 7.62 |
| _112 | +5° C. | 12 months | — | 9 | — | 10.37 | 2.38 | 0.89 | 0.97 | 0.31 |
| | +25° C./60% RH | 6 months | — | 9 | — | 10.03 | 2.37 | 0.89 | 2.07 | 2.04 |
| | +40° C./75% RH | 1 month | — | 9 | — | 10.03 | 2.31 | 0.89 | 3.22 | 5.71 |
| _113 | +5° C. | 12 months | — | 9 | — | 10.44 | 2.10 | 1.49 | 1.00 | 0.27 |
| | +25° C./60% RH | 6 months | — | 9 | — | 10.17 | 2.09 | 1.48 | 2.18 | 1.99 |
| | +40° C./75% RH | 1 month | — | 9 | — | 9.88 | 2.07 | 1.50 | 3.07 | 7.52 |

TABLE 4
Comparison of physical stability of the U300 formulations according to the invention (_114, _172, _173, _174)
against other U300 formulations

| | | | Composition | | | | | | | | |
|---|---|---|---|---|---|---|---|---|---|---|---|
| | | | Insulin | Sodium | | | | | Phosphate | Poly- | |
| | Storage | Storage | glulisine | chloride | Glycerol | m-Cresol | Phenol | Trometamol | dihydrate | sorbate 20 | Zinc |
| Formulation | condition | duration | [mg/mL] | [mg/mL] | [mg/mL] | [mg/mL] | [mg/mL] | [mg/mL] | [mg/mL] | [mg/mL] | [mg/mL] |
| _114 | +5° C. | 6 months | 10.48 | — | 9.5 | 2.1 | 1.5 | 6 | — | 0.01 | — |
| _172 | +5° C. | 6 months | 10.48 | — | 13.2 | 2.1 | 1.5 | 6 | — | 0.01 | — |
| _173 | +5° C. | 6 months | 10.48 | — | 11.3 | 2.1 | 1.5 | 6 | — | 0.01 | — |
| _174 | +5° C. | 6 months | 10.48 | — | 10.1 | 3.15 | — | 6 | — | 0.01 | — |
| _105 | +5° C. | 6 months | 10.48 | 5 | — | 3.15 | — | 6 | — | 0.01 | — |
| _106 | +5° C. | 6 months | 10.48 | 0.58 | 16 | 1.72 | 1.5 | — | 1.25 | — | 0.0196 |
| _107.1 | +5° C. | 6 months | 10.48 | 0.58 | 16 | 1.72 | 1.5 | — | 1.25 | — | — |
| _109 | +5° C. | 6 months | 10.48 | — | 10.2 | 2.1 | 1.5 | 6 | — | 0.01 | 0.0196 |
| _111 | +5° C. | 6 months | 10.48 | — | 10.0 | 2.4 | 1.5 | 6 | — | 0.01 | — |
| _112 | +5° C. | 6 months | 10.48 | — | 10.6 | 2.4 | 0.9 | 6 | — | 0.01 | — |
| _113 | +5° C. | 6 months | 10.48 | — | 10.3 | 2.1 | 1.5 | 6 | — | 0.01 | — |

| | | | Composition | | After 6 months long term storage at | | | |
|---|---|---|---|---|---|---|---|---|
| | Storage | Storage | Arginine | Methionine | +5° shaken at +37° C. with 120 rpm | | | |
| Formulation | condition | duration | [mg/mL] | [mg/mL] | T0 | T3 | T7 | T10 |
| _114 | +5° C. | 6 months | — | 9 | Clear, 1.15 FNU | Clear, 1.30 FNU | Clear, 1.28 FNU | Clear, 1.20 FNU |
| _172 | +5° C. | 6 months | — | 3 | Clear, 1.10 FNU | Clear, 1.13 FNU | Clear, 1.15 FNU | Clear, 1.19 FNU |
| _173 | +5° C. | 6 months | — | 6 | Clear, 1.11 FNU | Clear, 1.13 FNU | Clear, 1.14 FNU | Clear, 1.17 FNU |
| _174 | +5° C. | 6 months | — | 9 | Clear, 1.10 FNU | Clear, 1.16 FNU | Clear, 1.18 FNU | Clear, 1.20 FNU |
| _105 | +5° C. | 6 months | — | — | Clear, 1.95 FNU | Clear, 1.62 FNU | Clear, 1.97 FNU | Clear, 1.64 FNU |
| _106 | +5° C. | 6 months | — | — | Clear, 1.11 FNU | Slightly turibid, | Turbid, 64.90 | Turbid, 99.17 |
| | | | | | | 19.84 FNU | FNU | FNU |
| _107.1 | +5° C. | 6 months | — | — | Clear, 1.06 FNU | Slightly turibid, | Turbid, | Turbid, |
| | | | | | | 6.43 FNU | 28.17 FNU | 51.51 FNU |
| _109 | +5° C. | 6 months | 9 | — | Clear, 1.30 FNU | Clear, 1.28 FNU | Clear, 1.32 FNU | Clear, 1.31 FNU |
| _111 | +5° C. | 6 months | 9 | — | Clear, 1.41 FNU | Clear, 1.44 FNU | Clear, 1.47 FNU | Clear, 1.44 FNU |
| _112 | +5° C. | 6 months | 9 | — | Clear, 1.34 FNU | Clear, 1.35 FNU | Clear, 1.35 FNU | Clear, 1.36 FNU |
| _113 | +5° C. | 6 months | 9 | — | Clear, 1.37 FNU | Clear, 1.43 FNU | Clear, 1.58 FNU | Clear, 1.38 FNU |

### EXAMPLE 3

### Pharmacokinetics and Pharmacodynamics of U300 and U100 Formulations of Insulin Glulisine.

In castrated male Yucatan minipigs with a body weight of about 30 kg, diabetes mellitus was induced by treatment with alloxan about three week before the experiment. The alloxan-treated minipig is a model for type 1 diabetes mellitus in humans.

A first group of alloxan-treated minipigs (n=4) received 0.3 U/kg insulin glulisine U100 (100 U/mL) subcutaneously. The U100 composition corresponded to the commercial “Apidra” formulation. A second group (n=4) received 0.3 U/kg insulin glulisine U300 (300 U/mL) subcutaneously.

The plasma concentration of insulin glulisine was determined by a specific LC-MS/MS assay (detection level of 0.1 ng/mL). No difference was detected in the plasma concentration of insulin glulisine in U100 and U300 treated minipigs.

Upon treatment with U100 or U300 insulin glulisine, the glucose concentration in the plasma rapily decreased. No difference in the effect upon the plasma glucose was detected between the U100 and U300 group. In all animals of both treatment groups, the plasma glucose concentration was below the detection threshold within 3 hours after treatment.

This experiment demonstrates that an U300 formulation of insulin glulisine is suitable for the treatment of diabetes mellitus.

1: An aqueous pharmaceutical formulation comprising 200 U/mL of insulin glulisine.

2: The aqueous pharmaceutical formulation of claim 1 further comprising from 1 to 30 mg/mL methionine.

3: The aqueous pharmaceutical formulation of claim 1, wherein the aqueous pharmaceutical formulation comprises no more than 20 μg/mL of zinc.

4: The aqueous pharmaceutical formulation of claim 3, wherein the aqueous pharmaceutical formulation is essentially free of zinc.

5: The aqueous pharmaceutical formulation of claim 1 further comprising one or more of a buffer substance, a preservative, and a tonicity agent.

6: The aqueous pharmaceutical formulation of claim 5, wherein the buffer substance is trometamol.

7: The aqueous pharmaceutical formulation of claim 5, wherein the aqueous pharmaceutical formulation comprises 3 to 10 mg/mL trometamol.

8: The aqueous pharmaceutical formulation of claim 5, wherein the preservative is phenol and/or m-cresol.

9: The aqueous pharmaceutical formulation of claim 8, wherein the aqueous pharmaceutical formulation comprises 1.5 to 3.5 mg/mL m-cresol and/or 0.5 to 3.0 mg/mL phenol.

10: The aqueous pharmaceutical formulation of claim 5, wherein the tonicity agent is glycerol.

11: The aqueous pharmaceutical formulation of claim 10, wherein the aqueous pharmaceutical formulation comprises 5 to 26 mg/mL glycerol.

12: The aqueous pharmaceutical formulation of claim 1, wherein the aqueous pharmaceutical formulation is essentially free of phosphate.

13: The aqueous pharmaceutical formulation of claim 1 further comprising from 1 to 30 mg/mL arginine.

14: The aqueous pharmaceutical formulation of claim 1 further comprising a non-ionic surfactant at a concentration of 1 to 200 μg/mL, wherein the non-ionic surfactant is polysorbate 20, polysorbate 80, or poloxamer 171.

15: The aqueous pharmaceutical formulation of claim 1, wherein the pH is between 3 and 9.5, between 6 and 8.5, or between 7 and 7.8.

16: The aqueous pharmaceutical formulation of claim 1, wherein the aqueous pharmaceutical formulation is essentially free of chloride.

17: A method of treating a patient having type 1 diabetes mellitus or type 2 diabetes mellitus, comprising administering an effective amount of the aqueous pharmaceutical formulation of claim 1 to a patient in need thereof.

18: The method of claim 17, wherein the aqueous pharmaceutical formulation is administered to the patient by injection or an insulin pump.
